(19) United States
(12) Patent Application Publication
(10) Pub. No.: US 2012/0225848 A1
(43) Pub. Date: Sep. 6, 2012

(54) Antagonists of GABA-B Receptors to Enhance Neuronal Function, Learning and Memory

(76) Inventors: Alexander M. Kleschevnikov, Encinitas, CA (US); William C. Mobley, La Jolla, CA (US)

(21) Appl. No.: 13/497,234

(22) PCT Filed: Sep. 17, 2010
(86) PCT No.: PCT/US10/49380

§ 371 Date: May 23, 2012

Related U.S. Application Data
(60) Provisional application No. 61/277,205, filed on Sep. 21, 2009.

(51) Int. Cl. A61K 31/662; A61K 49/00; A61K 31/5375; A61P 25/00; A61K 31/185; A61K 31/137

(52) U.S. Cl. 514/114; 514/576; 514/654; 514/238.8; 800/3

(57) ABSTRACT

Methods for treating an individual to improve cognitive function are provided. In the subject methods, an effective amount of a GABAB antagonist, or a blocker of Kir3.2 potassium channels, is administered to the individual, resulting in an improvement in cognitive function of the host. The subject methods find use in a variety of different applications.

### FEDERALLY SPONSORED RESEARCH AND DEVELOPMENT

This invention was made with Government support under grants NS 38869, AG 16999 and HD 31498 awarded by the National Institutes of Health. The Government has certain rights in this invention.

### BACKGROUND OF THE INVENTION

Mental retardation (MR) affects 2-3% of the population in the industrialized world, and remains a prevalent form of non-progressive cognitive impairment. Defined by an IQ of less than 70 and deficits in academic, adaptive, and interpersonal skills, disorders involving MR are spread over a broad etiology, resulting from both genetic and non-genetic causes. The breadth and frequency of MR-related cognitive dysfunction is alarming considering that pharmacological intervention is currently non-existent.

Down syndrome (DS) is characterized by deficits in learning and memory. Mouse genetic models of DS have provided new perspectives on the genes responsible for DS-related changes. The Ts65Dn mouse is segmentally trisomic for chromosome 16 with an extra copy of ˜104 genes homologous to those on human chromosome 21. Ts65Dn mice recapitulate a number of physiological and behavioral features of DS. Recently it was reported that long-term potentiation (LTP), a cellular model for learning and memory, is impaired in the dentate gyrus (DG) of Ts65Dn mice, and that this change is related to enhanced efficiency of inhibition.

Which of the triplicated genes contribute to these abnormalities is not yet established. One of the genes providing a possible link between the DS-specific genetic abnormalities and the efficiency of inhibitory system is Kcnj6, which encodes for Kir3.2 (also referred to as Girk2) subunits of inwardly-rectifying potassium channels. Kir3.2 potassium channels serve as the major effectors for postsynaptic GABAB receptors (Luscher et al. (1997) Neuron 19, 687-695).

Metabotropic GABAB receptors play a prominent role in both inhibitory neurotransmission and synaptic plasticity. Activation of postsynaptic GABAB receptors results in generation of slow potassium-dependent inhibitory post-synaptic potentials and hyperpolarization of neurons. Activation of presynaptic GABAB receptors affects release of several neurotransmitters. Furthermore, selective antagonists of the GABAB receptors effectively modulate induction of LTP, and some exhibit properties of cognitive enhancers. However, no studies have addressed what role GABAB receptor signaling may play in hippocampal synaptic plasticity and excitatory/inhibitory balance in mouse models of DS.

The treatment of mental retardation, including DS, is of great clinical and humanitarian interest. The present invention addresses this issue. We showed that GABAB receptor antagonists restore LTP and improve learning and memory in a model of DS. GABAB antagonists also reduced the propensity for pro-epileptic activity in the neuronal circuits of the dentate gyrus. Thus, the results show that GABAB antagonists may improve learning and memory without provoking epilepsy in DS.

### PUBLICATIONS

US 2008-0009475 A1 discloses methods of treating Down Syndrome with antagonists of GABA-A.

The synaptic connections in the Ts65Dn brain have been assessed by a variety of assays. For example, quantitative electron microscopy (EM) of Ts65Dn CNS has revealed a loss of asymmetric, excitatory synapses in Ts65Dn cortex relative to WT tissue, with a concurrent sparing of symmetric, inhibitory synapses (Kurt et al., 2000). Reductions in the density of excitatory synapses, and in the ratio of excitatory-to-inhibitory signaling in the Ts65Dn brain, have been noted alongside compensatory increases in the synaptic apposition lengths of asymmetric and symmetric synaptic junctions.

Recent studies using lucifer-yellow filling of neurons in Ts65Dn acute slices have indicated that a widening of synaptic clefts may relate to the development of enlarged spines in Ts65Dn cortex (see Belichenko et al. (2004) J Comp Neurol. 480(3):281-98). In an in vitro system, deficits in Ts65Dn hippocampal LTP were shown to reverse upon application of picrotoxin (see Kleschevnikov (2004) J. Neurosci. 24(37):8153-60). Costa et al., (2005) Neur. Lett. 382:317-322 report deficits in hippocampal CA1 LTP induced by TBS but not HFS in the Ts65Dn mouse. Levkovitz et al. (1999) J. Neuroscience 19:10977-10984 discuss upregulation of GABA neurotransmission in the suppression of hippocampal excitability and prevention of long-term potentiation in transgenic superoxide dismutase-over-expressing mice.

It has been suggested that excessive immunoreactivity of the glutamine receptor GluR1 may be involved in degeneration of neurons and the early formation of senile plaques in Down syndrome, as tissue samples taken from the frontal lobes of patients with Down syndrome exhibit homeostatic elevations in GluR1 immunoreactivity (Arai et al. (1996) Pediatr. Neurol. 15:203-206).

### SUMMARY OF THE INVENTION

Methods are provided for improving the cognitive function of an individual suffering from mental retardation as a result of Down Syndrome (trisomy 21). The individual is administered an effective dose of a GABA_{B} receptor antagonist, for a period of time sufficient to improve cognitive function. Long term cognitive improvement can be obtained from the methods of the invention, which can persist after cessation of treatment. GABAB antagonists may increase synaptic plasticity without increasing proepileptiform activity. Also provided are kits for use in practicing the subject methods.

In another embodiment of the invention, methods are provided for screening drug candidates for effectiveness in treating cognitive impairment associated with mental retardation. Such methods may include screening assays with animal or cell models, and may include a comparison with a reference value obtained from known GABA_{B} receptor antagonists. Screening may be used to identify agents that selectively target specific cells to improve targeting specificity of the intervention.

### BRIEF DESCRIPTION OF THE FIGURES

FIG. 1. Enhanced level of Kir3.2 (Girk2) in the hippocampus of Ts65Dn mice. (A) Examples of the Western blots and (B) Quantification of the data for 2N and Ts65Dn hippocampal samples. Note a higher level of Kir3.2 protein in the Ts65Dn samples. The amount of GBR1a and GBR1b subunits of the GABAB receptors and actin, which was used as a reference protein, was not different in 2N and Ts65Dn samples.

FIG. 2. Increased postsynaptic GABAB/Kir3.2 signaling in granule cells of Ts65Dn DG. (A) The GABAB agonist baclofen (40 μM, Bac) evoked larger whole-cell currents in Ts65Dn cells. (B) The GABAB agonist baclofen produced a greater change of input resistance in granule cells of Ts65Dn DG. Top panel—examples of currents evoked by hyperpolarizing steps (40 ms, 10 mV) before (a, a′), during (b, b′) and after (c, c′) application of baclofen. Such pulses were used to measure input resistance. Bottom panel—representative traces for input resistance in 2N and Ts65Dn granule cells during bath application of baclofen (40 μM, Bac) and quantification of input resistance (Rin) changes during application of baclofen. The reduction of Rin was greater in Ts65Dn cells.

FIG. 3A-B. Enhancement of LTP by the GABAB antagonist CGP55845. In Ts65Dn DG both the low (0.1 μM) and the higher (1.0 μM) concentrations of the drug increased LTP. Note that only the higher concentration was effective in DG of control 2N mice. This may suggest higher sensitivity of Ts65Dn neural circuits to treatment with GABAB receptor antagonists.

FIG. 4 Enhancement of LTP by the GABAB antagonist CGP52432 (1.0 μM). At this concentration the drug increased LTP in Ts65Dn DG, but not in 2N DG.

FIG. 5. Enhancement of LTP by fluoxetine (10 μM). Besides being a serotonin reuptake inhibitor, fluoxetine is also an effective blocker of Kir3.2 channels (IC50 ˜10 μM, see Kobayashi et al., 2004). Thus, similar to GABAB receptor antagonists, fluoxetine disrupted postsynaptic GABAB/Kir3.2 signaling. Fluoxetine increased LTP in Ts65Dn, but not in 2N slices. This suggests that suppression of currents through the Kir3.2 effector channels of the GABAB receptors allows for induction of LTP in Ts65Dn DG. Note that a concomitant increase in the level of serotonin, if it happened during the application of fluoxetine, would not enhance, but rather suppress LTP in DG (see, e.g., Sakai and Tanaka, 1993).

FIG. 6. Mechanisms for LTP enhancement by GABAB antagonists: Suppression of GABAB receptors with CGP52432 (1 μM) increased tetanus-evoked field EPSPs. The increase was similar in 2N and Ts65Dn slices. Examples of tetanus-evoked field EPSPs before and during application of CGP52432. Stimulus artifacts are truncated. Horizontal lines with triangles mark the tetani. Graphs show averages of each ten consecutive pulses of a 50-pulse tetanization train normalized to the area of total tetanus-evoked fEPSP. This figure shows that suppression of the GABAB receptors increased depolarization of postsynaptic neurons during tetanus. Increased depolarization may improve activation of the NMDA receptors (see FIG. 7).

FIG. 7. Mechanisms for LTP enhancement by GABAB antagonists: The NMDA receptor-mediated component was measured as the difference between the responses recorded before and during application of APV (50 μM). These measurements were carried out before, and then during, application of CGP52432.The magnitude of the NMDA receptor-mediated component was then evaluated as the area under the curve for the interval marked by a horizontal dashed line. The NMDA receptor-mediated component of the tetanus-evoked responses was smaller in Ts65Dn, but it was restored to 2N levels by the GABAB antagonist CGP52432 (1 μM).

FIG. 8 Baseline field responses in 2N and Ts65Dn DG. (A) Examples of field responses during I-O measurement. (B) Quantification of the I-O measurements. The initial slopes of fEPSP, paired-pulse ratios (Slope2/Slope1) and the population spike amplitudes did not distinguish 2N and Ts65Dn slices. However, the paired-pulse ratio of the population spike amplitudes (PS2/PS1) was significantly smaller in the Ts65Dn DG. Mean±SEM; n=8-9; *p<0.01.

FIG. 9. Effects of GABAB antagonist on pro-epileptic properties in DG. ‘Paired-pulse depression’ of the population spikes was increased by CGP52432 (1 μM) to the same degree in 2N and Ts65Dn slices. ‘Paired-pulse depression’ was measured as the ratio of the second to first population spike amplitudes (PS2/PS1). The responses were evoked by paired stimuli with interstimulus interval 30 ms. Paired-pulse depression of population spike is a measure of feedback inhibitory efficiency. Decrease of the (PS2/PS1) ratio corresponds to an enhancement of the feedback inhibitory efficiency. The result thus suggests an enhancement of the feedback inhibition by the GABAB antagonist. Because feedback inhibition prominently affects excitation of neurons, this result suggests also that GABAB antagonists may reduce propensity of neuronal circuits in DG to pro-epileptiform activity.

FIG. 10A-B. Effect of GABAB antagonist CGP55845 (1 μM) on spontaneous ictal bursts in high-potassium model of epilepsy. In order to explore directly the impact of GABAB receptor antagonists on pro-epileptic properties in the dentate gyrus, we used high-potassium model of epilepsy. The baseline frequency of the ictal bursts in high-potassium media was greater in Ts65Dn slices, suggesting an enhanced propensity of trisomic mice to epilepsy. CGP55845 had no effect on frequency of the bursts, but reduced the amplitude of burst-associated field potentials. This effect was greater in Ts65Dn slices. The result thus suggests that GABAB antagonists may ameliorate seizures.

FIG. 11. Behavioral studies. The GABAB receptor antagonist CGP55845 (0.5 mg/kg, i.p injections daily) did not affect spontaneous locomotor activity. Activity was greater in Ts65Dn in both vehicle control (Veh) and CGP-treated (CGP) groups of mice. CGP55845 treatment showed no effect on the locomotor activity. Baseline=before drug; Acute=one week of treatment; Chronic=three weeks of treatment.

FIG. 12. Time course of the baseline parameters of spontaneous locomotor activity in ‘Activity Box’. Graphs on the right show the averaged data for the entire 10-min period. Mean±SEM; n=13-15; *p<0.05. (A) Total distance moved. (B) Velocity. (C) Ambulatory time. (D) Resting time. The activity was higher in Ts65Dn mice.

FIG. 13. Baseline parameters of locomotion measured in the Open field sub-regions. Mean±SEM; n=13-15; *p<0.01. (A) Schema of the open field sub-regions. (B) Total distance moved. (C) Velocity. (D) Maximum distance moved. (E) Frequency of entering in a sub-region. (F) Duration spent in each sub-region. Insert: the time spent in the arena center is shown enlarged. The activity was higher in Ts65Dn mice.

FIG. 14. GABAB antagonist CGP55845 (0.5 mg/kg, daily i.p injections during 3 weeks) improved hippocampus-dependent memory in novel object recognition test. (A) Time spent on investigating the objects during the acquisition trial was similar in all groups of mice. This suggests equal curiosity of 2N and Ts65Dn mice, and no effect of GABAB antagonists on exploratory habits. (B) Time spent on investigating the objects during the testing trial 24 hours later was similar in all groups of mice. The discrimination index was smaller in Ts65Dn Veh group, but restored in the Ts65Dn CGP group of mice. Thus, the result suggests that hippocampus-dependent recognition memory is impaired in Ts65Dn mice, and that treatment with GABAB antagonists restore the memory.

Table 1. Baseline behavioral parameters in activity box and open field. Ts65Dn mice were more active in both the activity box and the open field tests. This is evident from greater distance, velocity, ambulatory time and rearing frequency for the Ts65Dn mice, as compared to 2N diploid mice.

Table 2. Effect of GABAB antagonist CGP55845 on behavioral parameters in the ‘Activity box’. Suppression of GABAB receptors had no affect on any of the parameters measured in the 2N or Ts65Dn mice. Baseline=before administration of drug. Acute=after 1 week of drug treatment. Chronic=after 3 weeks of drug treatment. a—significant difference between 2N Veh and Ts65Dn Veh groups; b—significant difference between 2N CGP and Ts65Dn CGP groups;

Table 3. The GABAB antagonist CGP55845 had no effect on parameters measured in the Open Field testing. Ts65Dn mice were more active as evident from increased distance, velocity and rearing frequency. Injections of CGP5845 did not affect general activity of 2N nor Ts65Dn mice. Baseline=before administration of drug. Acute=after 1 week of drug treatment. Chronic=after 3 weeks of drug treatment. a—p<0.05 for 2N Veh vs Ts65Dn Veh; b—p<0.05 for 2N CGP vs Ts65Dn CGP.

### DESCRIPTION OF THE SPECIFIC EMBODIMENTS

The cognitive function of an individual suffering from mental retardation is alleviated by administration of a GABA_{B} receptor antagonist for a period of time sufficient to improve cognitive function. The dosing regimen is usually maintained for at least about one week, at least about two weeks, at least about three weeks, at least about one month, or more.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

γ-Aminobutyric acid (GABA) is the major inhibitory neurotransmitter in the mammalian central nervous system. The predominant effect of GABA is the interaction with specific receptor proteins which results in an increase of either chloride or potassium ion conductance of the post-synaptic membrane to produce an inhibition of neuronal firing. In recent years, much attention has been focused on specific GABA_{B} receptors linked to potassium channels.

As shown in the examples provided herein, decreased functionality in cognitive performance of an individual suffering from mental retardation can be associated with increased inhibition in the brain. Treatment by chronically administering drugs that reduce activity of post-synaptic GABA_{B} channels is shown to improve cognitive function. Compounds of interest specifically reduce the activity of GABA_{B} receptors or associated effector channels.

γ-Aminobutyric acid type B (GABA_{B}) receptors mediate the metabotropic actions of the inhibitory neurotransmitter GABA. These seven-transmembrane receptors are known to signal primarily through activation of G proteins to modulate the action of ion channels or second messengers. The functional GABA_{B} receptor is made up of a heterodimer consisting of two subunits, GABA_{B}-R1 and GABA_{B}-R2, which interact via coiled-coil domains in their C-terminal tails. GABA_{B} receptors couple through G proteins to effectors—K⁺ and/or Ca²⁺ channels. Activation of GABAB receptors stimulates the opening of K⁺ channels which brings the neuron closer to the equilibrium potential of K⁺, thus hyperpolarising the neuron. This prevents sodium channels from opening, action potentials from firing, and VDCCs from opening, and so stops neurotransmitter release. Thus GABAB receptors are considered inhibitory receptors.

Heterotrimeric guanine nucleotide-binding proteins (G proteins) are the transducers that convey information from agonist-occupied receptors to a variety of effector proteins. Included in the receptors coupled to G proteins are the GABA_{B} receptors. GPCRs, G proteins, and the effector ion channel can form stable complexes that persist during signal transduction in vivo.

Of particular interest for the present invention are GABA_{B} receptors coupled to Kir3.2 ion channels through G proteins. A variety of cells including neuronal cells possess inwardly rectifying K⁺ (Kir) channels through which currents flow more readily in the inward direction than outward. These K⁺ channels play pivotal roles in maintenance of the resting membrane potential, in regulation of the action potential duration, in receptor-dependent inhibition of cellular excitability, and in the secretion and absorption of K⁺ ions across cell membrane. Kir channels constitute a family of K⁺ channels whose subunits contain two putative transmembrane domains and a pore-forming region. It is a feature of the Kir channel family that each subfamily plays a specific physiological functional role.

In some embodiments of the invention an effective dose of GABA_{B} antagonist will at least transiently alter the potassium flow at Kir3.2 channels associated with GABA_{B} receptors in the central nervous system, i.e. for a period of at least about 1 minute, at least about 5 minutes, at least about 30 minutes, at least about 1 hours, or more, usually not more than about 4 hours; not more than about 3 hours; not more than about 2 hours.

However, there may also be embodiments where the effective dose provides for a longer lasting effect, for example where the dose, e.g. a very low dose, provides for a longer lasting alteration of channel activity coupled to GABA_{B} receptors in the central nervous system, e.g. for at least about 12 hours, at least about 24 hours, or longer.

GABA_{B} Antagonists. Antagonists of interest selectively block the signaling activity of a GABA_{B} receptor, e.g. a GABA_{B} receptor coupled to a Kir3.2 ion channel. The transient decrease in activity is usually at least about 50%, at least about 75%, at least about 90% or more. Antagonists of GABA_{B} are known in the art and available to those of skill in the art. Antagonists include CGP62349, CGP52432, CGP56999 and SCH50911 ((2S)(+)-5,5-dimethyl-2-morpholineacetic acid). For example, CGP 55845A at a concentration of 1 μM blocks baclofen (5-10 μM)-induced postsynaptic hyperpolarization and depression of evoked IPSPs and EPSPs.

Structures of selected antagonists are as follows:

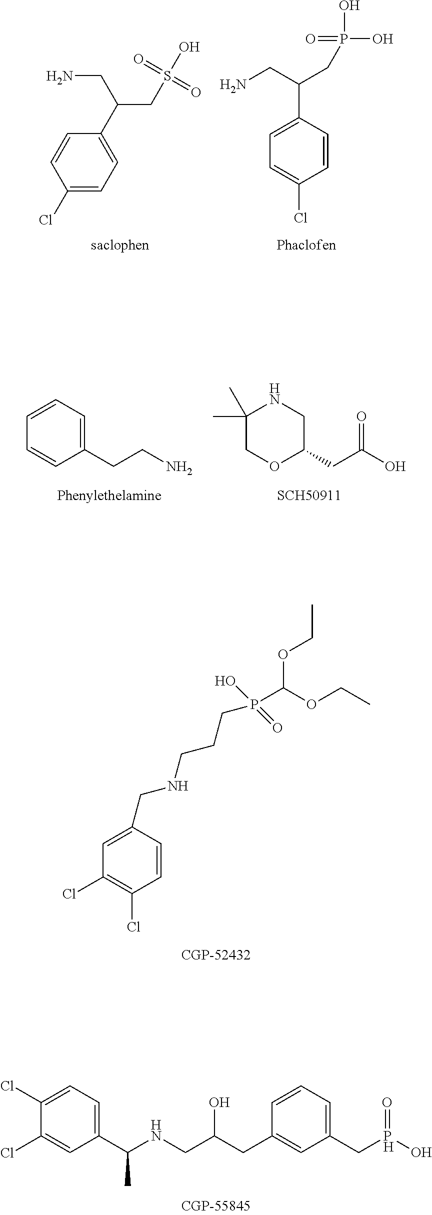

It is a feature of the invention that the antagonists are selective for GABAB receptors, i.e. there is substantially no activity at GABAA receptors, and the antagonist is generally non-epileptogenic.

### Conditions of Interest

Various forms of MR may be attributed to the over-inhibition of cortical circuits, resulting in major homeostatic disturbances in circuit activity that underlies learning and memory. By inhibition of GABA_{B} for an effective period of time, over-inhibition is relieved, allowing for long term changes to neuronal interactions. A number of conditions may be treated by the methods of the invention. Such conditions include, without limitation, those listed below.

Down Syndrome. Down syndrome is the most frequent genetic cause of mild to moderate mental retardation and associated medical problems and occurs in one out of 800 live births, in all races and economic groups. Down syndrome is a chromosomal disorder caused by the presence of an additional third chromosome 21 or “trisomy 21.” Three genetic variations can cause Down syndrome. In approximately 92% of the time, Down syndrome is caused by the presence of an extra chromosome 21 in all cells of the individual. In approximately 2-4% of cases, Down syndrome is due to mosaic trisomy 21, and the remaining cases result from a translocation trisomy 21.

Most people with Down syndrome have IQ's that fall in the mild to moderate range of retardation. Premature aging is a characteristic of adults with Down syndrome. In addition, dementia, or memory loss and impaired judgment similar to that occurring in Alzheimer disease patients, may appear in adults with Down syndrome. This condition often occurs when the person is younger than forty years old.

Observations of patients with DS suggest that imbalances in GABAergic and glutamatergic transmission, favoring a greater efficacy of GABAergic signaling, may be present during initial neurological developmental events. The methods of the invention demonstrate that targeted pharmacological intervention with GABA-B receptor antagonists can result in improvement in adult learning and memory.

Phenylketonuria is a mental retardation disorder caused by the deficiency of the hepatic enzyme phenylalanine-4 hydroxylase and the build-up of CNS phenylalanine. L-phenylalanine at concentrations observed in untreated PKU depresses the amplitude and frequency of both NMDA and non-NMDA components of mEPSP's in dissociated cortical cultures. Mechanistically, these effects are mediated in large part by phenylalanine's competitive antagonism of the obligatory agonist site of the NMDA receptor, but may involve other postsynaptic and presynaptic mechanisms as well. Golgi studies performed on children with PKU reveal a prevalence of immature dendritic spines in pyramidal cells of the cerebral cortex.

Neonatal Protein Malnutrition. Non-genetic forms of cognitive impairment can be induced by protein or caloric malnutrition. Morphological hallmarks of excessive inhibition can be observed in the cerebral cortex of malnourished individuals, with the proliferation of unusually long, narrow spines.

Fragile X Syndrome. MR syndromes brought about by specific deficits in neuronal signal transduction provide evidence for excessive inhibition as a major contributing factor to cognitive dysfunction. Fragile X syndrome is due to a trinucleotide repeat expansion in the FMR1 gene that prevents expression of its encoded protein product--fragile X mental retardation protein (FMRP). X-linked mental retardation associated with marXq28, or fragile X syndrome, is characterized by moderate to severe mental retardation, macroorchidism, large ears, prominent jaw, and high-pitched jocular speech. Expression is variable, with mental retardation being the most common feature.

Cortical cultures in an animal model of Fragile X syndrome display delayed formation and maturation of neuronal network activity, and decreased BDNF expression compared to cultures prepared from wild-type (WT) littermates. Complementing these electrophysiological findings are studies documenting a higher density of unusually long dendritic spines in fragile X patients.

Neuroimaging studies using fMRI have shown that FMRP levels are positively correlated with activation of the prefrontal cortex in individuals with fragile X during performance of a working memory task. These results suggest that FMRP is required during especially demanding cognitive exercises, and that failure to meet these demands with appropriate concentrations of FMRP result in decreased network activity. FMRP's role as a regulator of site-specific protein translation in dendritic spines may account for many of the observations that have been made in fragile X patients and in animal models of the disorder.

Neurofibromatosis 1. This condition is attributed to genetic mutations in the NF1 gene and loss of function of neurofibromin's ras guanosine triphosphatase (rasGAP) activity, presents the most direct link between overinhibition in the brain and mental retardation. Animals carrying a heterozygous null mutation of the NF1 gene (Nfl.sup.+/−) exhibit spatial learning deficits in the Morris water maze that intimately relate with increases in GABA-mediated inhibition. Nf1^{+/-} mice have larger mIPSP's and evoked IPSP's than WT controls, and decreases in hippocampal LTP. Thus, partial loss of neurofibromin's rasGAP activity, and subsequent unregulated ras activation, leads to abnormally high GABA-mediated inhibition, which underlies impairments in Hebbian plasticity and learning and memory. This devastating cascade of events can be prevented by administration of farnesyl transferase inhibitors, anti-ras agents, which return learning and memory in Nf1.sup.+/− adult mice to control levels.

Maple Syrup Urine Disease is a mental retardation disorder resulting from the loss of function of the branched chain L-α-keto acid dehydrogenase complex and a subsequent accumulation of the metabolic substrates a-ketoisocaproic acid (KIC), α-keto-β-methylvaleric acid (KMV), and α-ketoisovaleric acid (KIV). Experiments have shown that α-keto acids dampen cortical excitation and reduce learning in a dose-dependent fashion. Administration of physiologically relevant concentrations of KIV to dissociated cortical neurons significantly reduces spontaneous network activity, while intra-hippocampal infusion of KIC, KMV and KIV severely disrupts the acquisition of an inhibitory avoidance task. The effects of α-keto acids on cortical activity and cognition appear to be mediated via direct interactions of the metabolites with the vesicular glutamate transporter. Application of α-keto acids inhibits glutamate uptake into synaptic vesicles in a competitive manner and changes the chloride dependence for the activation of vesicular glutamate transport. α-keto acid inhibition of the vesicular glutamate transporter is dramatic during the acute phase of MSUD. Young children with MSUD demonstrate changes in neuronal morphology, exhibiting a conspicuous abundance of long, thin dendritic spines in the cerebral cortex.

Autism, often referred to as autistic disorder or infantile autism, is a complex behavioral disorder which, by definition, develops prior to age three years. Autism is defined completely on the basis of impairments in social interaction, impairments in communication, and repetitive and stereotypic behaviors. For most children, the onset of autism is gradual; however, approximately 30% have a “regressive” onset. Fifty to seventy percent of children with autism are defined as mentally retarded by nonverbal IQ testing. Seizures develop in approximately 25% of children with autism.

The standard diagnostic criteria for autism, compiled by the American Psychiatric Association Manual of Psychiatric Diseases, 4th edition (DSM-IV), are the primary diagnostic reference used in the United States. The causes of autism can be divided into “idiopathic,” which comprises the majority of cases, and “secondary,” in which an environmental agent, chromosome abnormality, or single-gene disorder can be identified.

The standard diagnostic criteria include qualitative impairment in social interaction, as manifested by at least two of the following; qualitative impairments in communication; stereotyped and repetitive use of language or idiosyncratic language; lack of varied, spontaneous make-believe play or social imitative play appropriate to developmental level; restricted repetitive and stereotyped patterns of behavior, interests, and activities. Criteria also include delays or abnormal functioning in at least one of the following areas, with onset prior to age three years: social interaction, language as used in social communication, or symbolic or imaginative play.

Impairment in social interaction separates individuals with autism from the people around them. Children with autism are unable to “read” other people, ignoring them and often strenuously avoiding eye contact. Most children with autism fail to develop reciprocal communication either by speech, gestures, or facial expressions. Deficits in pragmatic skills are present throughout life and affect both language and social interaction. In contrast to the child with nonspecific mental retardation or a primary developmental language disorder, who usually has better receptive than expressive language, the child with autism has impaired receptive language. Fifty to seventy percent of autistic children are defined as mentally retarded by nonverbal IQ testing.

Children with Down syndrome have autism more commonly than expected. The incidence was at least 7% in one study. This finding suggests that chromosome abnormalities may lower the threshold for the expression of autism.

Whereas a very small percentage of children with autism have fragile X syndrome, at least half of children with fragile X syndrome have autistic behaviors, including avoidance of eye contact, language delays, repetitive behaviors, sleep disturbances, tantrums, self-injurious behaviors, hyperactivity, impulsiveness, inattention, and sound sensitivities.

One of the DSM-IV-defined pervasive developmental disorders, Rett syndrome exhibits considerable phenotypic overlap with autism; children with both disorders often have a period of normal development followed by loss of language with stereotypic hand movements. Decreasing rate of head growth over time and hand-wringing in female individuals may suggest the diagnosis of Reft syndrome. Molecular genetic testing for MECP2 mutations that cause Rett syndrome is clinically available. Only 1% of individuals with the diagnosis of autism have been reported to have a MECP2 coding region mutation, however these two disorders may be causally related based on reports of variants in the 3′-UTR of MECP2 in three of 24 individuals with autism and variable MeCP2 expression in the brains of individuals with both Rett syndrome and autism.

### Assessment

By mental retardation is meant a cognitive impairment with a pattern of persistently slow learning of basic motor and language skills during childhood, and a significantly below-normal global intellectual capacity as an adult. One common criterion for diagnosis of mental retardation is a tested intelligence quotient (IQ) of 70 or below. Conditions of interest for treatment include Down Syndrome, and other congenital or acquired conditions that impair cognitive function. Included in the conditions of interest for treatment are those in which there is impairment, often from early childhood, of at least one cognitive function, such as a impairment in memory, impairment in learning ability, etc.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as impairment in memory or learning ability or other cognitive function. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

As mentioned above, in these applications an effective amount of GABA_{B} receptor antagonist is administered to the host. By “effective amount” is meant a dosage sufficient to produce a desired result, where the desired result is generally an amelioration or alleviation, if not complete cessation, of one or more symptoms of the disease being treated, particularly the cognitive impairment symptoms, e.g., memory, learning ability, and the like.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In addition to the above methods of treatment, the subject methods also find use in the prophylactic or preventative treatment regimens. In such methods, the host is administered an amount of a direct GABA_{B} inhibitor, typically according to a dosage schedule (e.g., daily, weekly, monthly etc.), that is sufficient to prevent the occurrence of at least symptoms of the disorder, e.g., impaired cognition.

In the treatment of a patient, assessment will usually include a clinical history and the collection of standardized information. Assessment may also include IQ testing. In animal models, a variety of standardized tests may be utilized for evaluation of learning and memory. Examples include analysis of sustained and non-sustained attention and impulsivity, e.g. acquisition inhibitory avoidance responding; 5-choice serial reaction time testing in rodents and a distractor version of a Delayed Match to Sample test in monkeys. Analysis of social and working memory may include novel object recognition model, social recognition model; spatial working memory using a water maze; and spontaneous alternation ‘T’ and ‘Y’ mazes. Mazes, e.g. water maze with a hidden platform; 2-choice visual discrimination water maze; “dry land” Barnes circular maze; etc. are useful in testing spatial reference memory. Different configurations of the water maze measure different forms of learning and utilize different brain systems. A second commonly used paradigm for studying learning and memory is the conditioned fear test. The direct measure of freezing behavior in response to discrete conditioned stimuli such as tones or lights as a measure of learning can evaluate two discrete forms of learning, cued and contextual. A passive avoidance model is useful in assessing recall.

Many assessment tests are available. For example, memory, attention and executive function (planning abilities) can be assessed by direct testing with the participants using the DAME battery. The DAME battery has been validated as a measure that is sensitive to change in older people with Down's syndrome. The range of scores is 0-241 and can be completed in 45 minutes by most people with mild-moderate learning disability.

Independent functioning can be evaluated using the Adaptive Behavioural Scale (ABS, Nihira, 1974). This is an informant based instrument and is part of the assessment used by the American Association on Mental Deficiency to assess daily living skills in people with learning disabilities. The ABS measures ten groups of skills related to self-care and socialization. The ten skills groups: independent functioning, physical development, economic activity, language development, numbers and time, domestic activity, vocational activity, self-direction, responsibility, and socialization.

The Clinician's Global Impression of Change (CGI/C) has been one of the most commonly used tests to assess overall change in clinical trials. The validity of this type of measure is based on the ability of an experienced clinician to detect clinically relevant against trivial change in a patient's overall clinical state.

In certain situations, treatment according to the subject methods results in a complete removal of a deficit in the cognitive function. The amount of improvement is at least about 2 fold, usually at least about 5 fold and more usually at least about 10 fold as compared to a suitable control, e.g., an otherwise substantially identical host not administered a GABA_{B} receptor antagonist, e.g., a host having similar level of cognitive ability that has been administered a placebo, where in certain embodiments the amount of improvement is at least about 25 fold, 50 fold, 75 fold, 100 fold or greater. The cognitive function improvement can be evaluated using any convenient protocol, where suitable protocols include, but are not limited to: Wechsler Adult Intelligence Scale (WAIS_-R) [Wechsler, D. WAIS-R Manual. New York: Psychological Corporation, 1981; Mini-Mental State Examination (MMSE) [Folstein et al. Mini Mental State: a practical method for grading the cognitive state of patients for the clinician. J Psychiat Res 1975; 12:189-98; Information-Memory-Concentration test; Fuld Object Memory Evaluation (FOSE) [Fuld, P A. The Fuld Object Memory Test. Chicago: The Stoeltimg Instrument Company, 1981]; The Buschke Selective Reminding Test (BSRT) [Buschke, H. Selective reminding for analysis of memory and learning. J Verbal Learn Verb Behav 1973; 12:543-50]; The Rey Auditory Recall Test [Buschke, H. Selective reminding for analysis of memory and learning. J Verbal Learn Verb Behav 1973; 12:543-50]; The Beton Visual Retention Test (BVRT) [Benton, A L. The revised visual attention test, 4.sup.th edn. New York: Psychological Corporation, 1974]; The California Verbal Learning Test [Delis et al. The California Verbal Learning Test. New York: Psychological Corporation, 1987]; Assessment of navigation in humans [Maguire et al. Knowing where and getting there; a human navigation network [Science 1998; 280:921-924]; and the like.

### Methods

Methods are provided for improving a cognitive function in a mammalian host. The host is generally a mammal, e.g. mouse, rat, monkey, etc. and in many embodiments is a human. The GABA_{B} receptor antagonist, or a blocker of Kir3.2 potassium channels, is administered at regular intervals, usually at least weekly, more usually daily, or every two days, and may include a sleep period between doses. Typically, the active agent is fast acting, and after administration the antagonist reaches therapeutic levels across the blood brain barrier at least transiently, e.g. for around about 1 minute, at least about 5 minutes, at least about 30 minutes, at least about 1 hour, or more. It is not believed to be necessary to maintain such levels throughout the treatment period. The agent may be short lived, where half-life in the blood is less than about 4 hours, less than about 3 hours, less than about two hours.

Administration of the treatment is maintained for a period of time sufficient to effect a change in cognitive function. Such treatment may involve dosing for at least about one week, at least about two weeks; at least about 3 weeks; at least about one month; at least about two months; at least about four to six months; or longer, for example at least about one or more years. For extended treatment; e.g. treatment of one or more years, a schedule may involve intermittent periods, such as one week on and one week off; two weeks on and two weeks off; one week in a month, etc.

Patients that can benefit from the present invention may be of any age and include adults and children, e.g. young adults. Children, e.g. neonate, infant, early childhood, adolescent, etc. in particular may benefit prophylactic treatment. Children suitable for prophylaxis can be identified by genetic testing for predisposition, e.g. by chromosome typing; by family history, or by other medical means. As is known in the art, dosages may be adjusted for pediatric use.

The GABA_{B} receptor antagonist is generally administered to the host as a pharmaceutical composition that includes an effective amount of the GABA_{B} receptor antagonist in a pharmaceutically acceptable vehicle. In the subject methods, the active agent(s) may be administered to the host using any convenient means capable of resulting in the desired improvement on cognitive function.

Therapeutic agents can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

Many GABA_{B} receptor antagonists are known to be bioactive in the central nervous system after oral or parenteral administration. For those that are not, one strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant for example.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain. Intrathecal administration maybe carried out through the use of an Ommaya reservoir, in accordance with known techniques. (F. Balis et al., Am J. Pediatr. Hematol. Oncol. 11, 74, 76 (1989).

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In another aspect of the invention, candidate agents are screened for the ability to improve cognitive impairment. Such compound screening may be performed using an in vitro model, a genetically altered cell or animal, or purified protein, particularly the human GABA_{B} receptor or cells expressing such a receptor. A wide variety of assays may be used for this purpose. In one embodiment, compounds that are active in binding assays with the channel proteins, or are predicted to be antagonists of the receptor are then tested in an in vitro culture system. Alternatively, candidate agents are tested for Kir3.2 blocking activity, and may then be assessed in animal models for treatment of cognitive impairment. Drug testing may further assess the activity of a compound in kindling epilepsy, so as to exclude epileptogenic compounds.

For example, candidate agents may be identified by known pharmacology, by structure analysis, by rational drug design using computer based modeling, by binding assays, and the like. Such candidate compounds are used to contact cells in an environment permissive GABA_{B} channel function. Such compounds may be further tested in an in vivo model for improvement of cognitive impairment.

The term “agent” as used herein describes any molecule, e.g. protein or pharmaceutical, with the capability of modulating cognitive impairment by acting through neuronal inhibitory pathways. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of GABA_{B} inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Compounds that are initially identified by any screening methods can be further tested to validate the apparent activity. The basic format of such methods involves administering a lead compound identified during an initial screen to an animal that serves as a model for humans and then determining the effects on cognitive impairment. The animal models utilized in validation studies generally are mammals. Specific examples of suitable animals include, but are not limited to, primates, mice, and rats.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

The following examples are offered by way of illustration and not by way of limitation.

### Experimental

### Abnormal Signaling Through GABAB Receptors in Dentate Gyrus Contributes to Failed Synaptic Plasticity in the Ts65Dn Mouse Model of Down Syndrome

Down syndrome (DS), a genetic disorder due to trisomy for chromosome 21, features cognitive impairment that prominently involves hippocampus. In the dentate gyrus (DG) of Ts65Dn mice, a genetic model for DS, we showed previously that long-term potentiation (LTP) was significantly reduced and that this abnormality is linked to enhanced inhibition. One link between DS, the inhibitory system and synaptic plasticity involves metabotropic GABAB receptors. Postsynaptic GABAB receptors use as effectors potassium channels containing Kir3.2 subunits, the gene for which (Kcnj6) is present in three copies in DS.

It is shown herein that signaling through postsynaptic GABAB receptors is altered, and that this abnormality contributes to failed LTP in Ts65Dn DG. It was found that the Kir3.2 level is increased in the Ts65Dn hippocampus. The GABAB agonist baclofen evoked larger whole-cell currents and produced a greater reduction of input resistance in Ts65Dn granule cells, confirming that GABAB/Kir3.2 signaling is stronger in the Ts65Dn DG. Suppression of GABAB receptors with CGP52432 allowed for induction of LTP in Ts65Dn DG. Interestingly, suppression of GABAB receptors resulted also in enhancement of feedback inhibition, and in reduction of amplitude of spontaneous ictal discharges in high-potassium model of epilepsy. These results show that GABAB receptor signaling plays an important role in synaptic plasticity and inhibitory/excitatory balance in Ts65Dn DG. Moreover, GABAB antagonists can increase synaptic plasticity without increasing proepileptiform activity.

### Materials and Methods

Animals. Segmental trisomy 16 (Ts65Dn) mice were obtained by mating female carriers of the 17 chromosome (B6EiC3H-a/A-Ts65Dn) with (C57BL/6JEi×C3H/HeJ)F1 (JAX #JR1875) males (Davisson et al., 1993). Ts65Dn mice are thus maintained on the B6/C3H background. To distinguish 2N from Ts65Dn mice, tail samples were used to extract genomic DNA; a quantitative polymerase chain reaction protocol (provided by the Jackson Laboratory) was used to measure M×1 gene expression. M×1 is present in three copies in Ts65Dn. Ts65Dn and 2N control mice were housed 2 to 5 per cage with a 12 h light-dark cycle and ad lib access to food and water. Experimental mice were males 3 to 4 months old.

Slice preparation. Mice were anesthetized with isoflurane before decapitation. The brain was quickly removed and immersed for 2 min in ice-cold artificial cerebrospinal fluid (ACSF) containing 119 mM NaCl, 2.5 mM KCl, 2.5 mM CaCl₂, 1.3 mM MgSO₄, 1 mM NaH₂PO₄, 26 mM NaHCO₃, 10 mM glucose, osmolarity 310, continuously bubbled with 95% O₂-5% CO₂, pH 7.4. The hippocampus was extracted and cut in ice-cold ACSF with a vibratome (Leica 1000) into 350-μm-thick transverse slices, which were allowed to recover in oxygenated ACSF at room temperature for at least 2 h prior to experimental recordings. In experiments using whole-cell recordings, slices were collected in a solution containing 250 mM sucrose, 2.5 mM KCl, 1.3 mM CaCl₂, 2.5 mM MgSO₄, 1 mM NaH₂PO₄, 26 mM NaHCO₃, and 10 mM glucose (Moyer and Brown, 1998).

Recording of evoked field potentials. A slice was transferred into the recording submerged chamber and superfused with ACSF at a constant rate of 2.5 ml/min at 32° C. Recording electrodes were made of borosilicate glass capillaries (1B150F, World Precision Instruments, Sarasota, Fla.) and were filled with 2M NaCl (resistance 0.3-0.5 MΩ). Monopolar stimulating electrodes were maid of Pt/Ir wires with diameter 25.4 μm (PTT0110, World Precision Instruments, Sarasota, Fla.) and had 100-μm-long exposed tips. The stimulating electrode was inserted under visual control perpendicular to the slice surface into the middle molecular layer (MML), and the recording electrode into the granule cell layer of the DG upper blade. The distance between the electrodes was 250-300 μm. If not otherwise specified, testing stimuli evoked field responses with population spike amplitudes 65-75% of maximum.

The amplitude of the population spike was measured as follows: 1) a line was drawn at the base of the population spike connecting the first and second peaks of the field response; 2) a second line was drawn at the peak of the population spike (i.e. at the peak of the downward deflection); 3) at the peak of the spike, a line was drawn vertically between these two lines to give the amplitude of the population spike. The magnitude of the field excitatory postsynaptic potential (fEPSP) was measured as the initial slope of the linear part of the fEPSP (latencies 0.1-0.9 ms). One pair of test stimuli (interstimulus interval 30 ms) was applied every 2 min. LTP was induced by tetanization with three trains of stimuli (0.5 s×100 Hz; 10 s between the trains).

Whole-cell recordings. Whole-cell recordings were performed from DG granule cells using an Axoclamp-2A amplifier (Axon Instruments, Union City, Calif.). In studies of whole-cell currents evoked by baclofen, the recording electrodes were filled with a solution containing (in mM): 125 K-Gluconate, 10 KCl, 0.1 CaCl₂, 1 EGTA, 10 mM HEPES, 2 MgATP, 0.2 Na₂-GTP, osmolarity 295, pH 7.3. The holding potential was −80 mV. Input resistance was measured by applying a hyperpolarizing step (40 ms, 15 mV) from a holding potential of −80 mV every 10 s throughout the experiment. Access resistance was monitored during the recordings. The data were discarded if the changes in the access resistance were greater than 15%.

Chemicals. CGP52432 was purchased from Tocris Cookson Inc. (Ellisville, Mo.). All other chemicals were purchased from Sigma-Aldrich (St. Louis, Mo).

Western Blot. The hippocampi from 2N and Ts65Dn mice were dissected on an ice-cold preparation table and homogenized in RIPA buffer (50 mM Tris-HCl, 1% NP-40, 0.25% Nadeoxycholate, 150 mM NaCl, 1 mM EDTA, 1 mM PMSF, 1 mM Na₃VO₄, 1 mM NaF) with 1 mg/ml protease inhibitor cocktail (aprotinin, leupeptin, pepstatin). The protein concentrations were determined using BCA protein Assay kit (Pierce, Rockford, Ill.). β-actin was used as a reference protein. 14 μl (1 mg/ml) of total protein per lane were loaded onto 4-12% Bis-Tris gels (Invitrogen, Carlsbad, Calif.), and separated by electrophoresis at 80 volts for ˜3 hours. Proteins were transferred to PVDF (polyvinylidene fluoride) microporous membranes, and the membranes were blocked with 4% nonfat milk in TBS-T solution (20 mM Tris-HCl, 150 mM NaCl, 0.1% Tween-20, pH 7.6). Membranes were then incubated with antibodies against Kir3.2 (1:200, Upstate, USA), GBR1 (1:1000, Chemicon, USA) or actin (1:1000, Upstate, USA). The blots were washed in TBS-T (3 times×10 min) followed by incubation with goat antirabbit IgG-HRP conjugates at a dilution of 1:10,000. The blots were washed in TBS-T and then developed with SuperSignal (Pierce, Rockford, Ill.). Immunoblots were scanned by a scanner Duoscan f40 (AGFA, NJ), and the images analyzed by program ImageJ (NIH, USA).

Behavioral testing. All mice were exposed to the same series of behavioral tests starting at 2-2.5 months of age. Each mouse was handled for 10 min, twice a day, during the 7 days that preceded testing and for 3 days in between tests. Tests for spontaneous locomotor activity and open field were performed before the drug, during the acute (1 week), and during the chronic (3 weeks) phases of the drug administration. Novel object recognition task with a 24 hours delay was performed during the chronic phase (3 weeks) of the drug administration. All behavioral testing took place during the light cycle between 7:00 a.m. and 7:00 p.m. and was performed at room temperature (22° C.). On the day of testing, the mice were left in their home cages in the experimental room for 2 hours for habituation. To minimize olfactory cues from the previous trial, each apparatus was thoroughly cleaned with 10% ethanol after each animal. CGP55845 (0.5 mg/kg) or equivalent volume of saline was injected intraperitoneally once a day during 3 weeks. Body weight was measured weekly. The body weight was lower in Ts65Dn than in 2N mice, and it was not affected by the treatment (Supplementary FIG. S6). All behavioral tests and procedures were performed by operators who were blind to the genotypes and drug treatment.

Spontaneous locomotor activity. Spontaneous locomotor activity was monitored using square Plexiglas activity chambers (43.2×43.2×20 cm) and activity monitor software (Med Associates Activity Monitor, version 5.93.773). The activity chambers were mounted with three planes of infrared detectors, within specially designed sound-attenuating chambers (66×55.9×55.9 cm). The animal was placed in the center of the testing arena under bright ambient light conditions and allowed to freely move for 10 minutes while being tracked by an automated tracking system. Distance moved, velocity, resting time, and times spent in pre-defined areas of the arena were recorded. At the conclusion of each trial the surface of the arena was cleaned with 10% ethanol.

Open field activity. Open field activity was recorded with a video camera in the arena of white squared box (76×76×50 cm) and analyzed with an automated videotracking system EthoVision Pro 3.1 (Noldus Information Technology, Wageningen, The Netherlands). Mice were placed in the center of the open field arena under dim ambient light conditions and activity was monitored for 10 min in a single trial. At the conclusion of each trial the surface of the arena was cleaned with 10% ethanol. Results were averaged for total distance traveled, number of entries and the time spent in the pre-determined areas (the center of the arena, the periphery, i.e. area extending 12 cm from the center, and the border, which included the four corners).

Novel object recognition. The Bevins and Besheer protocol (Bevins and Besheer, 2006) for two sample objects with one environment was used to study learning and memory with 24 hours delays in 2N and Ts65Dn mice. Before testing, mice were habituated in a black Plexiglas chamber (31×24×27 cm) during 10 min for 2 consecutive days under dim ambient light conditions. Activity of mice during the object recognition task was recorded with a video camera. Two objects (A and B) consistent with height and volume, but different in shape and appearance were used in this test. During the first day, two identical objects were placed diagonally across the two corners of the chamber. A mouse was placed at the mid-point between the objects. After 10 min exploring the objects, the mouse was return to the colony. To test for object recognition, 24 hours later one familiar object and one novel object were placed in the chamber and the mouse was again placed in the chamber for 3 min to explore the objects. The amount of time spent exploring each object (nose sniffing and head orientation within <1.0 cm) was recorded and evaluated by an operator, who was blind to both the phenotype and the treatment. The discrimination ratio was computed as R=Tnew*100/(Tnew+Told), were Tnew and Told is the time spent exploring the new and the old object respectively.

Statistics. For all experiments, data are presented as mean±sem (n is number of slices). In behavioral tests, two-way ANOVA followed by Turkey's post hoc test was used. In other comparisons, Students T-test was used and p<0.05 was taken as statistically significant.

### Results

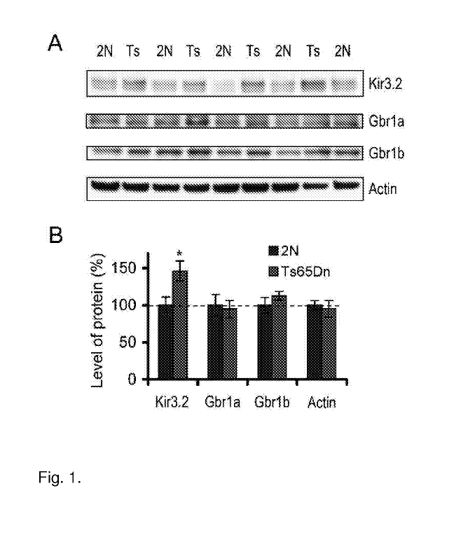

Increased level of Kir3.2 (Girk2) in the hippocampus of Ts65Dn mice. Ts65Dn mice have three copies of Kcnj6, the gene that encodes the Kir3.2 subunit of a G protein-coupled inwardly rectifying potassium channel. Using Western blot analysis, we examined whether or not the presence of an extra copy of Kcnj6 is translated into an increased level of the Kir3.2 protein (FIG. 1). FIG. 1A shows an immunoblot with samples from the hippocampi of individual 2N and Ts65Dn mice. As is evident, the level of Kir3.2 subunits (line 43 kDa) was consistently higher in the Ts65Dn samples. Quantitation showed that, when expressed with respect to 2N values, the Kir3.2 level was increased by about 50% in Ts65Dn mice (2N: 100±7.6%, n=5; Ts65Dn: 146.2±13.2%, n=4, p<0.01) (FIG. 1B). Actin (line 42 kDa), which was used as the reference protein, was also not different in 2N and Ts65Dn samples (2N: 100±6.2%, n=5; Ts65Dn: 95.2±11.3%, n=4, p>0.6). Similarly, expression of GBR1a (line 130 kDa) and GBR1b subunits (line 105 kDa) were not changed in the hippocampus of Ts65Dn mice (e.g., GBR1b: 2N: 100±17.3%, n=5; Ts65Dn: 103.1±18.0%, n=4, p>0.5). Thus, the level of Kir3.2 protein was increased in proportion to gene dose, while the level of GABAB receptor subunits GBR1a and GBR1b was not changed in the hippocampus of Ts65Dn mice.

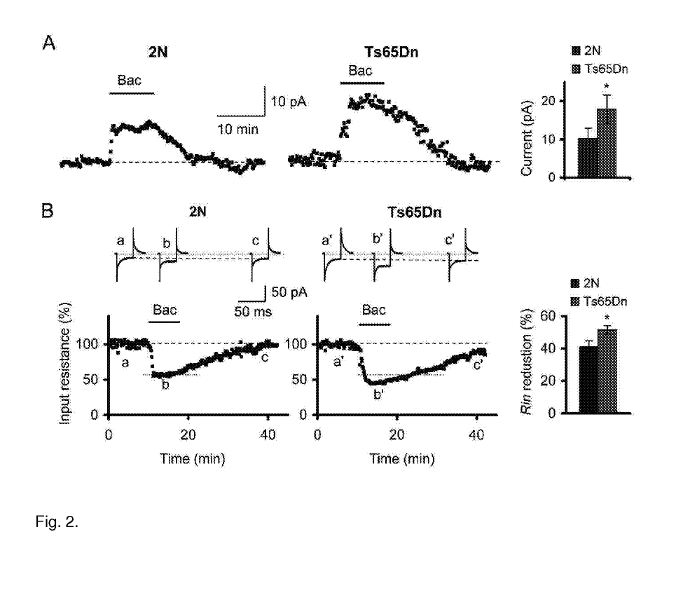

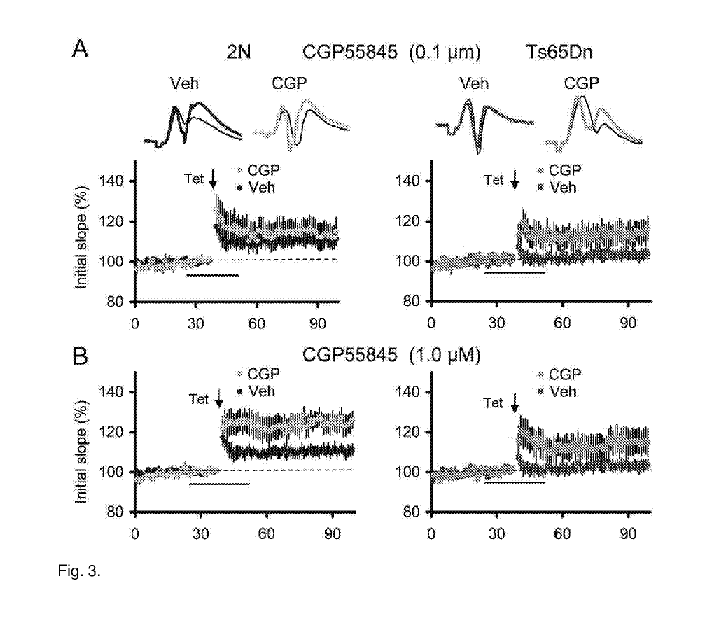

Enhanced function of postsynaptic GABAB receptors in Ts65Dn granule cells. Since Kir3.2-containing potassium channels serve as major effectors of postsynaptic GABAB receptors, the increased level of Kir3.2 in the Ts65Dn hippocampus raised the possibility of increased function of postsynaptic GABAB receptors. To test this prediction, we measured whole-cell currents and the input resistance of DG granule cells during bath application of the selective GABAB agonist baclofen (40 μM). Application of baclofen resulted in the generation of outward currents and a reduction of the input resistance (FIG. 2). The changes were greater in Ts65Dn than in 2N cells. On average, the whole-cell current evoked by application of baclofen was approximately 75% greater in Ts65Dn than in 2N cells (2N: 10.2±2.7 pA, n=7; Ts65Dn: 17.9±3.7 pA, n=7, p<0.05) (FIG. 2A). Similarly, the reduction of the input resistance was greater in Ts65Dn than in 2N cells (2N: 41.01±3.62%, n=7; Ts65Dn: 51.31±2.72%, n=7, p<0.03) (FIG. 2B). Thus, increased expression of Kir3.2 is accompanied by increased function of postsynaptic GABAB receptors in the Ts65Dn DG.

Membrane potential was more negative in Ts65Dn granule cells. Postsynaptic Kir3.2-containing potassium channels have a role in regulating resting membrane potential. Since the equilibrium potential for potassium ions is more negative (˜−90 mV) than the resting membrane potential (˜−75 mV), enhanced conductivity of potassium channels hyperpolarizes the neuron. An increase in the number of Kir3.2-containing potassium channels in Ts65Dn cells would be expected to result in hyperpolarization. We compared the resting membrane potential in granule cells of 2N and Ts65Dn DG; it was significantly more negative in Ts65Dn than in 2N cells, registering a difference of approximately 6 mV on average (2N: −72.7±2.2 mV, n=19; Ts65Dn: −78.4±2.2 mV, n=11, p<0.05). The values of the input resistance were not statistically different (2N: 409.8±39.1 MΩ, n=9; Ts65Dn: 363.3±32.6 MΩ, n=8, p>0.35). We conclude that enhanced expression of Kir3.2 is accompanied by increased hyperpolarization of the granule cells in Ts65Dn DG.

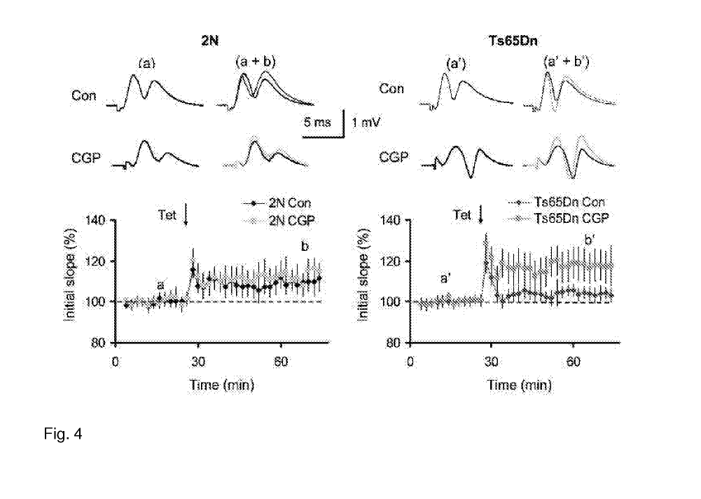

Suppression of GABAB receptors allowed for induction of LTP in the Ts65Dn DG. Enhanced signaling through postsynaptic GABAB receptors in the Ts65Dn DG would be expected to reduce depolarization of neurons during tetanus and contribute to deficient LTP. If so, suppression of the GABAB receptors with selective antagonists would increase depolarization and restore LTP. We examined the effects of GABAB antagonists on LTP in a series of experiments. First, we examined the effect of both low and high concentrations of the GABAB antagonist CGP55845 (0.1 μM and 1.0 μM respectively). At the low concentration, CGP55845 enhanced LTP in Ts65Dn, but not in 2N slices (FIG. 3A). At the higher concentration, CGP55845 enhanced LTP in both Ts65Dn and 2N slices (FIG. 3B). If, as predicted (Pozza et al., 1999), the low concentration CGP55845 suppressed mainly postsynaptic GABAB receptors (see Discussion), the results suggest that suppression of the postsynaptic GABAB receptors is sufficient to enhance LTP in the Ts65Dn DG. Next we tested effect of another selective GABAB receptor antagonist CGP52432 (1 μM). This drug also enhanced LTP in the Ts65Dn DG (FIG. 4).

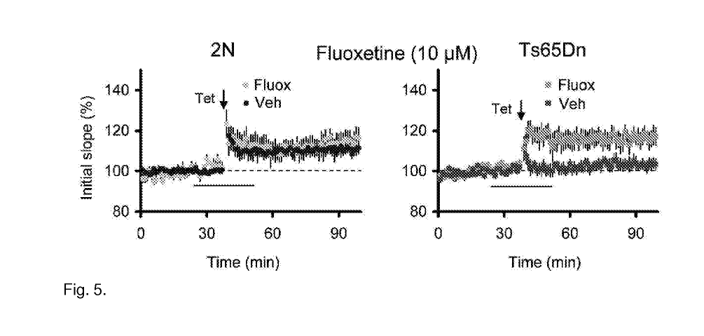

To examine further the role of the postsynaptic GABAB/Kir3.2 signaling on LTP, we tested the effect of fluoxetine. Best known as a serotonin-reuptake inhibitor, fluoxetine effectively suppresses currents through Kir3.2-containing potassium channels with an 1050 value of ˜10 μM (Kobayashi et al., 2004). We found that fluoxetine (10 μM) enhanced LTP in the Ts65Dn DG (FIG. 5). The LTP induced in Ts65Dn DG in the presence of fluoxetine was comparable to that seen with the GABAB antagonists. We conclude that suppression of postsynaptic GABAB receptors, or their effector channels, improves synaptic plasticity in the Ts65Dn DG.

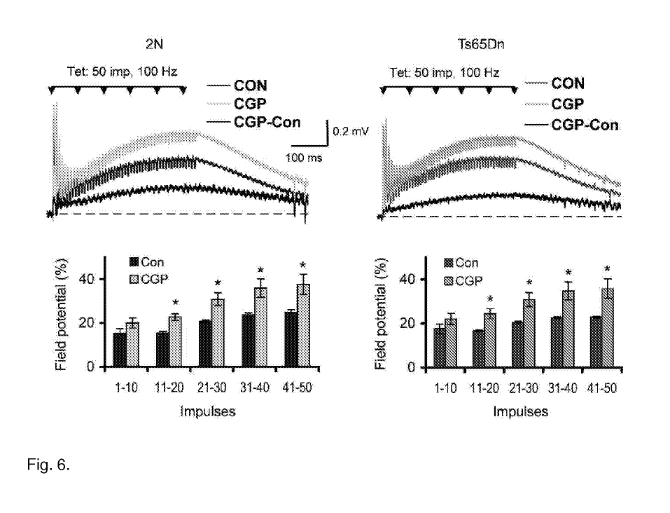

A possible mechanism for LTP enhancement by GABAB antagonists. One mechanism by which GABAB antagonists could increase LTP would be through greater depolarization of neurons during tetanus, allowing for better activation of NMDA receptors. To test this possibility, we first examined the effect of CGP52432 (1 μM) on field responses during tetanizations (FIG. 6). Suppression of GABAB receptors significantly increased the tetanus-evoked field responses (p<0.01); the effect was equal in 2N and Ts65Dn slices. Because the amplitude of field responses reflects depolarization during tetanus, these data indicate that suppression of the GABAB receptors enhances depolarization, thus likely increasing activation of the NMDA receptors.

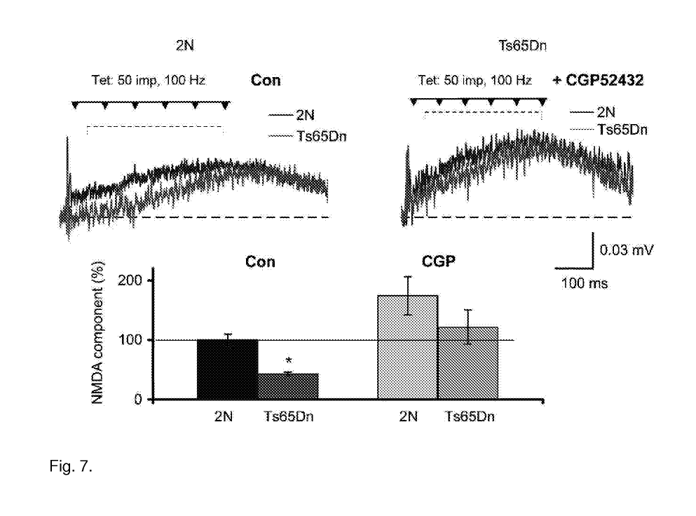

We next examined the effect of CGP52432 on the NMDA receptor-mediated component of the tetanus-evoked field responses, which approximates activation of the NMDA receptors by tetanus (FIG. 7). The NMDA receptor-mediated component was measured as the difference between the responses recorded before and during application of APV (50 μM). These measurements were carried out before, and then during, application of CGP52432 (1 μM). Because the first tetanization affected the responses by inducing LTP, two tetanizations with a 10-min interval were applied, and the response evoked by the second tetanus was used for analysis. Before CGP application, the NMDA-receptor mediated component was smaller by ˜60% in Ts65Dn slices (p<0.01) (FIG. 7). Application of CGP enhanced the NMDA receptor-mediated responses and eliminated the difference between 2N and Ts65Dn slices (p=0.69) (FIG. 7). We conclude that GABAB antagonists act to restore LTP in Ts65Dn DG by increasing depolarization of neurons during tetanus, allowing for more effective activation of NMDA receptors.

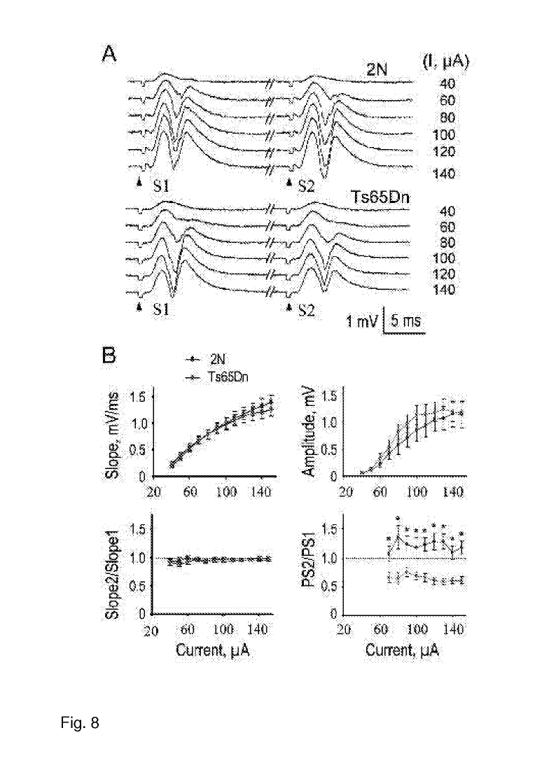

Effects of GABAB antagonists on pro-epileptic properties in Ts65Dn DG. People with DS show an increased risk of epilepsy. Any treatment to improve learning and memory in DS must be tested for potential adverse effects on inducing or exacerbating the risk of seizures. Feedback inhibition in the DG prominently affects both induction of LTP and neuronal excitation. We evaluated the effect of GABAB antagonists on feedback inhibition by measuring paired-pulse depression of the population spike amplitude (PS2/PS1). Baseline parameters of excitatory neurotransmission, including initial slope of fEPSPs, paired-pulse facilitation (fEPSP2/fEPSP1) and PS amplitude were similar in 2N and Ts65Dn slices (FIGS. 8A, B). Consistent with earlier observations (Kleschevnikov et. al., 2004), the PS2/PS1 ratio was lower in Ts65Dn DG (FIG. 8B); this finding suggests increased feedback inhibition in Ts65Dn slices.

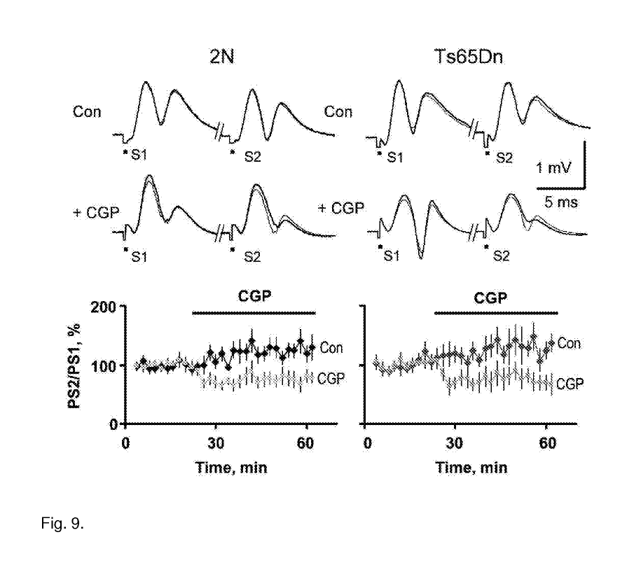

Application of CGP52432 (1 μM) reduced the PS2/PS1 ratio in both 2N (from 1.09±0.18 to 0.93±0.25) and Ts65Dn (0.71±0.16 to 0.59±0.17) slices (FIG. 9). Scaled to pre-drug baseline values, the changes were similar (p=0.45). These data are evidence that GABAB antagonists increase the efficiency of the feedback inhibitory system both in the 2N and Ts65Dn DG. Increase of feedback inhibition may restrict excitability of neurons, thus reducing propensity of neural circuits to pro-epileptic activity.

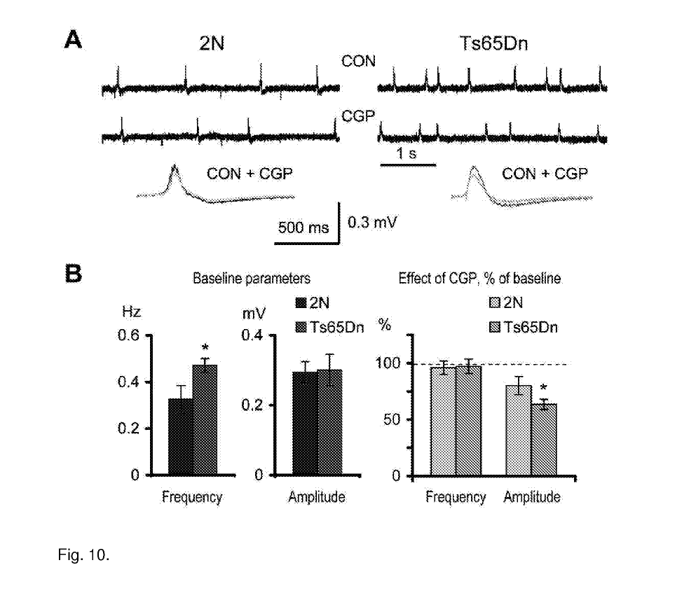

To evaluate more directly the potential effects of GABAB antagonists on epileptic properties of DG neural circuits, we examined spontaneous ictal bursts in hippocampal slices provoked by an increase in extracellular potassium. Increasing extracellular potassium from 2.5 mM to 7.5 mM resulted in spontaneous ictal bursts in both 2N and Ts65Dn DG (FIG. 10). The baseline frequency of the bursts was greater in Ts65Dn (p<0.03), suggesting an increased propensity of these mice to show pro-epileptiform activity. Application of the GABAB antagonist CGP55845 (1 μM) did not affect the frequency of bursts (p=0.8), but significantly reduced the amplitude of burst-associated field potentials (p<0.01) (FIGS. 10A, B). These changes were greater in Ts65Dn than in 2N slices (p<0.05). GABAB antagonists may thus possess antiepileptic potential in this model of ictogenesis. While the mechanism of the effect is yet to be refined, reduced electrochemical potential for potassium ions and the enhancement of feedback inhibition may both play a role.

Behavioral testing. Enhancement of LTP by GABAB receptor antagonists raised the possibility that these drugs may improve learning and memory in Ts65Dn mice. To test this, we examined the effect of the GABAB antagonist CGP55845 (0.5 mg/kg, i.p. daily, 3 weeks) on hippocampus-dependent memory using the novel object recognition test. In addition, spontaneous locomotor activity and open field activity were tested before the treatment, and during acute (1 week), and chronic (3 weeks) phases of the treatment.

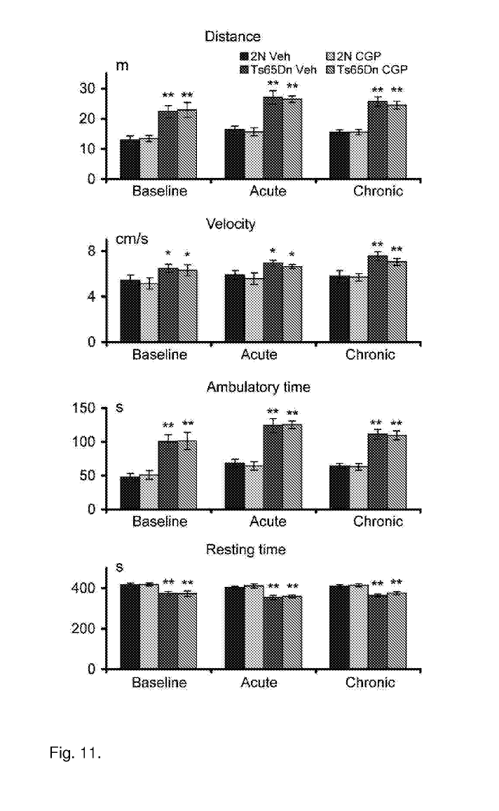

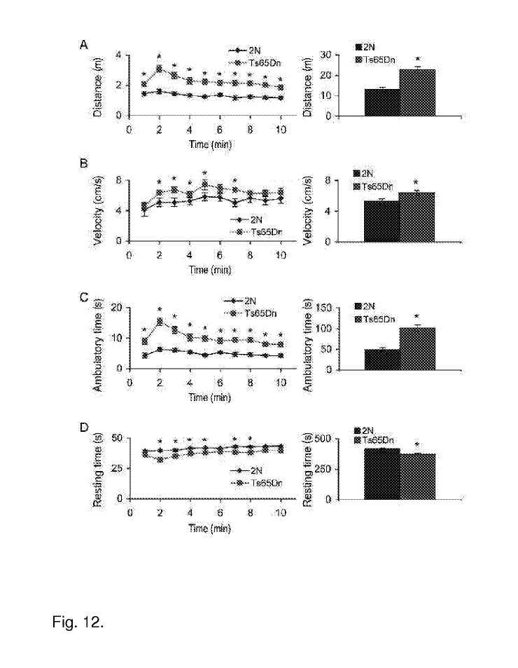

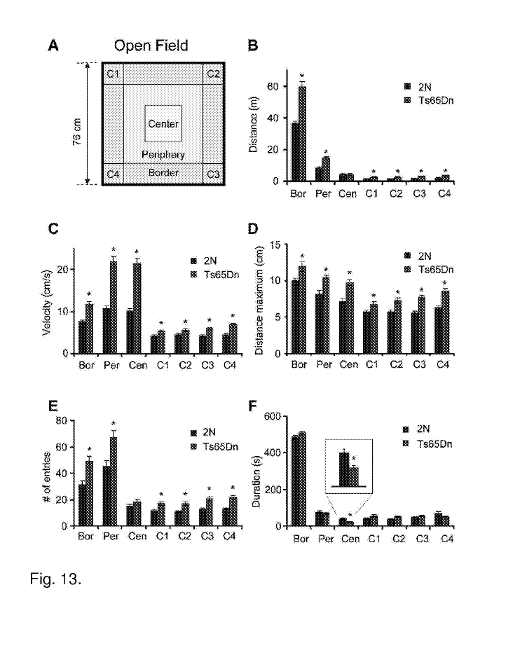

Spontaneous locomotor activity. Testing in the ‘activity box’ showed that baseline locomotor activity was significantly higher in Ts65Dn than in 2N control mice (FIGS. 11 and 12; Tables 1 and 2). Average velocity, total distance traveled and the number of ambulatory episodes were greater, while the resting time was shorter in Ts65Dn mice (Table 2; FIG. 12). Treatment with the GABAB receptor antagonist CGP55845 did not affect spontaneous locomotor activity (FIG. 11; Table 2). We conclude that suppression of the GABAB receptors does not affect locomotor activity in 2N and Ts65Dn mice.

Open field. Testing in the open field allows estimation of general activity, anxiety and exploratory habits of animals. Similar to the results in activity box, average velocity and total distance traveled were significantly greater in Ts65Dn mice (Table 1; FIG. 13). We observed also that 2N and Ts65Dn mice spent equal time in most regions of the field except the arena center, in which Ts65Dn mice spent significantly less time (Table 1; FIG. 13F). Treatment with CGP55845 had no significant effect on any of these parameters (Table 3).

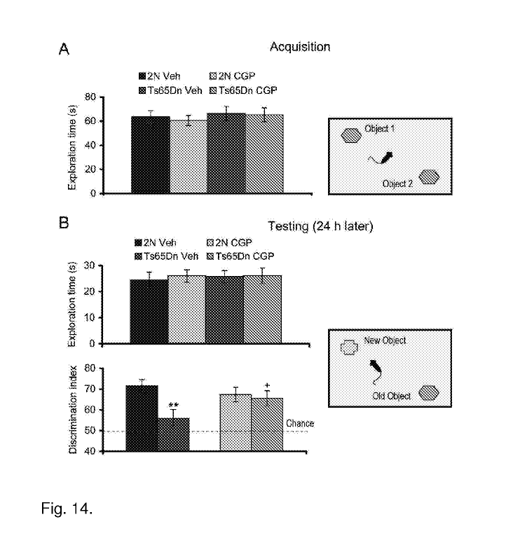

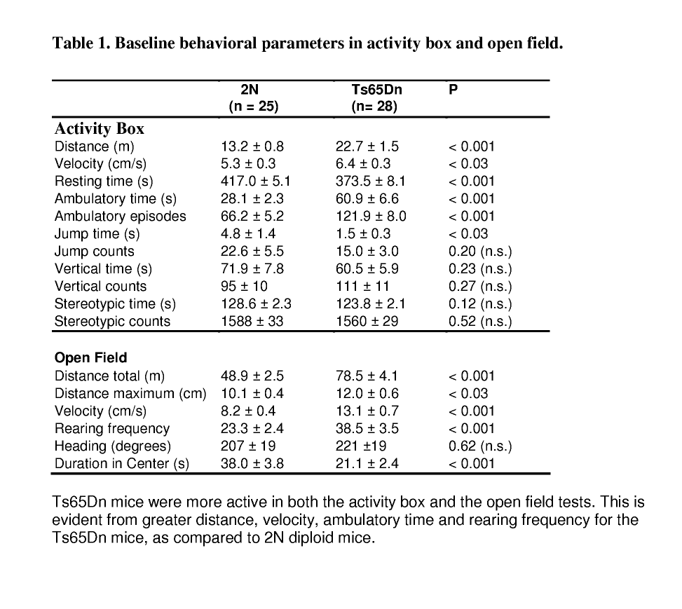

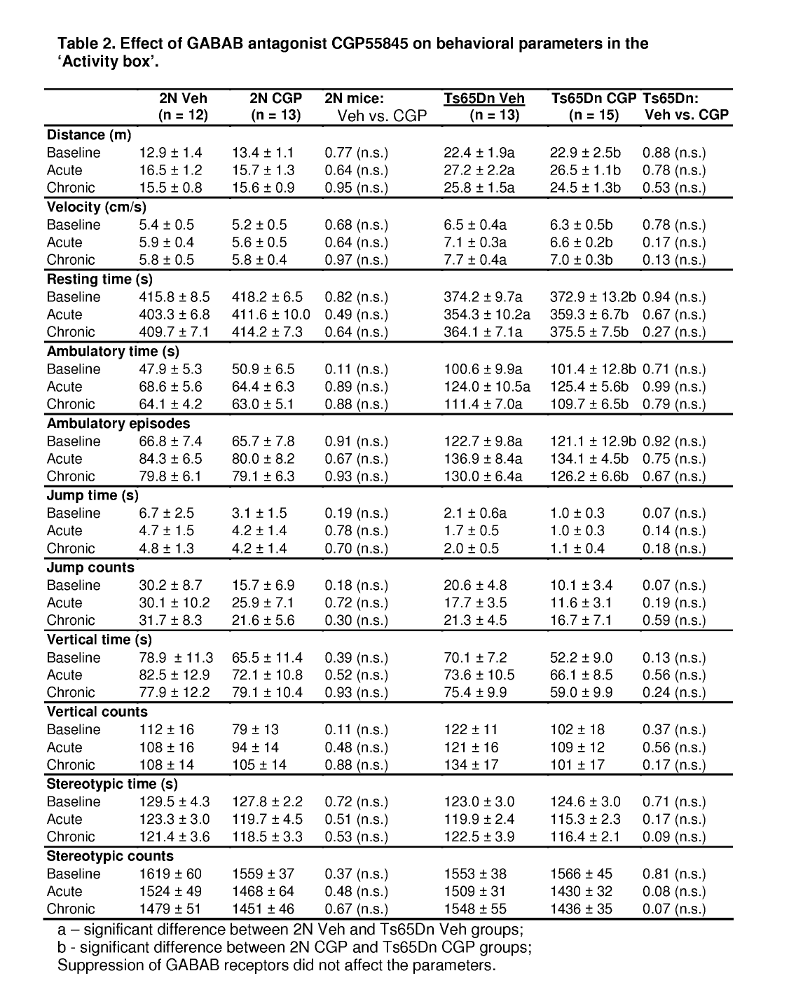

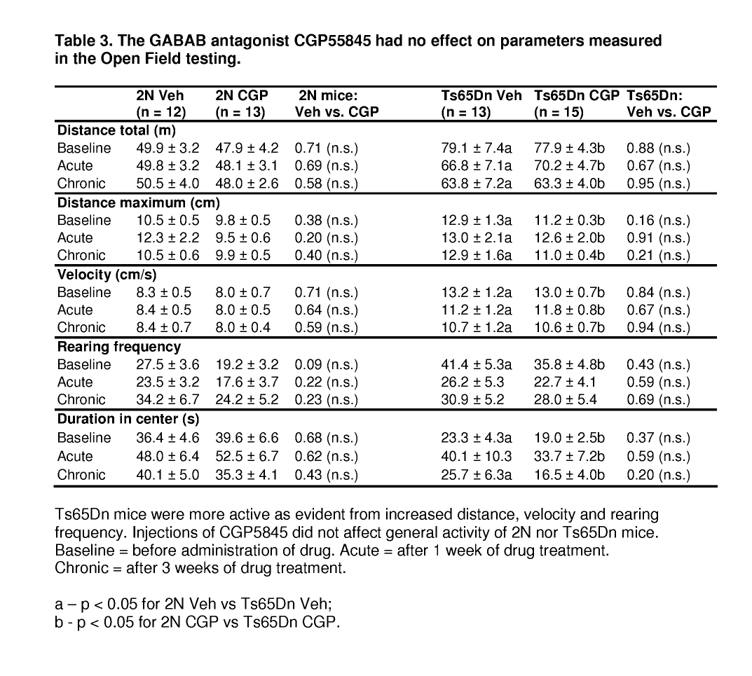

Novel Object Recognition. To assess hippocampus-dependent recognition memory we used a ‘Novel object recognition’ test with a 24-h interval between the acquisition and testing phases. As shown, this type of memory is severely impaired in mouse models of DS (Fernandez et al., 2007; Belichenko et al., 2009b). On day one of the testing the mouse was placed in the center of the acquisition box and allowed to investigate two similar objects. All groups of mice spent, on average, equal time investigating the objects (p=0.4-0.9) (FIG. 14A), suggesting that 2N and Ts65Dn mice had a similar degree of curiosity and that treatment with the GABAB antagonist did not affect exploration habits. On day two, one of the objects was replaced with a new object and the mice were allowed to explore again. Total time of exploration was again equal for all groups of mice (p=0.45-0.9) suggesting no effect of CGP55845 on exploration habits. However, Ts65Dn mice treated with vehicle control showed a severe abnormality of memory. Indeed, with respect to 2N mice they spent more time investigating the old object and less time investigating the novel object. This was reflected in a significantly lower discrimination index for the Ts65Dn mice (p<0.01) (FIG. 14B). Treatment with CGP55845 improved performance of Ts65Dn mice. The discrimination index of the CGP-treated Ts65Dn mice was significantly higher then in the Ts65Dn vehicle control mice (p<0.05), and it was not different from the discrimination index in the 2N CGP mice (p=0.7) (FIG. 14). Thus, suppression of GABAB receptors improved hippocampus-dependent recognition memory in Ts65Dn mice.

DS results in several phenotypes reflecting cognitive dysfunction. Herein we show that the increased dose of Kcnj6 acting through the enhanced efficiency of postsynaptic GABAB receptors restore deficits in learning and hippocampus-dependent memory in Ts65Dn mice. We report that: 1) the Kir3.2 protein is increased in the Ts65Dn hippocampus; 2) signaling through postsynaptic GABAB receptors is more efficient in the Ts65Dn DG; 3) Suppression of the GABAB/Kir3.2 signaling, using two different GABAB receptor antagonists and a blocker of Kir3.2 channels, markedly increased LTP in the Ts65Dn DG; 4) the smaller NMDA receptor-mediated component of field responses during tetanus in Ts65Dn was restored by a GABAB antagonist; and 5) GABAB antagonists improved hippocampus-mediated memory. Remarkably, treatment with GABAB antagonists was not accompanied by an enhancement of pro-epileptiform activity, a characteristic potentially important for future attempts to pursue the therapeutic potential of these molecules in DS. In fact, the evidence show that GABAB antagonists appear to enhance feedback inhibition and reduce the amplitude of ictal bursts spontaneously generated in high-potassium media. We conclude that antagonists of GABAB receptors restore synaptic plasticity in the DG and may be effective as a treatment for learning and memory in individuals with DS.

Increased signaling through postsynaptic GABAB receptors. KCNJ6 is located in the middle of the so-called ‘Down syndrome critical region’ on human chromosome 21; in the mouse the region is located on chromosome 16. The protein product of Kcnj6 is the Kir3.2 (Girk2) subunit of G protein-activated inwardly-rectifying potassium channels, which serve as the major effector for GABAB and other postsynaptic metabotropic receptors (Luscher et al., 1997). The Kir3.2 protein is present at relatively high levels in hippocampus and neocortex (Murer et al., 1997). Thus, Kir3.2 provides a possible link between DS-specific genetic alterations and changes in inhibitory circuits in the DG. We observed that the level of the Kir3.2 protein was increased by ˜50% in the hippocampus of Ts65Dn mice, a finding consistent with earlier studies (Harashima et al., 2007). Thus, as is the case with many other products of triplicated genes (e.g. Salehi et al., 2006), gene dose predicts the level of the encoded protein. To test whether or not a relatively small increase in Kir3.2 would have a physiological consequence, we measured several physiological parameters. Because resting membrane potential is strongly influenced by currents through Kir3.2-containing potassium channels (Luscher et al., 1997; Koyrakh et al., 2005), we measured this parameter and observed a significantly more negative resting potential in granule cells of the Ts65Dn DG. This finding is consistent with increased function of Kir3.2-containing potassium channels.

Kir3.2 is preferentially localized to the extrasynaptic membrane of dendrites (Kulik et al., 2006). A number of metabotropic receptors use Kir3.2 channels as effectors; in addition to GABAB receptors, these include muscarinic m2, serotoninergic 5-HT1A, adrenergic α2 and other receptors (Dascal, 1997; Luscher et al., 1997; Mark and Herlitze, 2000). The subcellular distribution of GABAB receptors is very similar to that of Kir3.2 (Kulik et al., 2003). Postsynaptic GABAB receptors and Kir3.2 show a considerable degree of co-localization in extrasynaptic locations around putative glutamatergic synapses, where their presence may impact excitatory neurotransmission (Kulik et al., 2006). Whether or not an increase in Kir3.2 channels potentiates signaling through the metabotropic GABAB receptors is an important question. To explore directly the efficiency of the postsynaptic GABAB/Kir3.2 signaling, we applied the selective GABAB agonist baclofen and noted significantly greater whole-cell currents and a larger reduction of input resistance in Ts65Dn DG cells, an observation consistent with increased signaling. These results agree with an earlier finding that baclofen effects on current flow were increased in Ts65Dn primary cultured neurons (Best et al., 2007). We conclude that signaling through postsynaptic GABAB receptors is enhanced in the Ts65Dn DG.

GABAB antagonists improve LTP in Ts65Dn DG: possible mechanisms. Within the hippocampus, GABAB receptors are present not only at GABAergic synapses on granule cells, but also on inhibitory interneurons (Charles et al., 2003; López-Bendito et al., 2004) and on the presynaptic terminals of glutamatergic, GABAergic, somatostatinergic, and other axons (Davies and Collingridge, 1996; Raiteri 2008). Since each of these pathways plays a role in hippocampal function, it is virtually impossible to predict what overall effect GABAB-selective agonists or antagonists would have on synaptic properties. For example, suppression of postsynaptic GABAB receptors on granule cells may reduce cellular hyperpolarization and increase activation of the NMDA receptors, thus leading to greater LTP. In contrast, suppression of presynaptic GABAB receptors on GABAergic terminals or postsynaptic receptors on GABAergic interneurons could increase inhibitory drive, thus suppressing LTP. The plurality of loci for GABAB receptor expression within neuronal circuits may explain the apparent inconsistencies in studies using GABAB antagonists in normal rodents in which both facilitatory (Olpe and Karlsson, 1990; Olpe et al., 1993b; Staubli et al., 1999; Helm et al., 2005) and inhibitory (Davies et al., 1991; Brucato et al., 1996; Davies and Collingridge, 1996) effects on LTP were noted.

To investigate whether or not increased GABAB/Kir3.2 signaling in Ts65Dn impacts synaptic plasticity, we examined LTP. Two strategies were employed. First, we examined the effect of the GABAB antagonist CGP55845 at two different concentrations (0.1 μM and 1.0 μM). GABAB antagonists have several fold (˜5-7 times) greater potency for postsynaptic than for presynaptic receptors (Pozza et al., 1999). For CGP55845, the reported 1050 values for post- and presynaptic receptors in hippocampal slices are 0.11 μM and 0.74 μM respectively (Pozza et al., 1999). Thus, at the lower concentration of CGP55845 used, one would expect to suppress mostly postsynaptic receptors; at the higher concentration, both post- and presynaptic receptors would be affected. LTP in Ts65Dn DG was equally enhanced by both concentrations of CGP55845. This suggests that suppression of postsynaptic GABAB receptors was sufficient to restore LTP in Ts65Dn, and that additional suppression of the presynaptic receptors had little effect.

Second, we examined effect of a Kir3.2 channel blocker on LTP. Fluoxetine, a serotonin reuptake inhibitor, is also an effective blocker of the Kir3.2-containing potassium channels (Kobayashi et al., 2004). We found that fluoxetine enhanced LTP in Ts65Dn slices, thus supporting the idea that changes in postsynaptic GABAB/Kir3.2 signaling affect LTP. It should be noted that a concomitant increase in serotonin due to application of fluoxetine would not enhance LTP in DG. On the contrary, increased release of serotonin would likely reduce LTP (e.g. Sakai and Tanaka, 1993).

To understand better the mechanisms of LTP enhancement by GABAB antagonists, we measured the NMDA receptor-mediated component of field responses during tetanus. It was significantly smaller in the untreated Ts65Dn DG, thus providing a plausible link to reduced LTP. Pointing to a role for GABAB receptors in suppressing NMDA currents, GABAB antagonist increased the NMDA receptor-mediated component. In Ts65Dn slices it was increased to the level seen in the untreated 2N slices. We conclude that one mechanism by which antagonists of the GABAB receptors increased LTP was through enhanced depolarization during tetanus, allowing for better activation of the NMDA receptors.

Interestingly, in the 2N DG only the higher concentration of CGP55845 enhanced LTP; the lower concentration of CGP8845 and fluoxetine were both relatively ineffective. This suggests a limited potential for suppression of postsynaptic GABAB/Kir3.2 signaling in enhancement of LTP in normal diploid mice. A number of additional mechanisms can be envisioned to explain facilitatory effects of GABAB antagonists on synaptic plasticity in normal animals. First, suppression of presynaptic GABAB receptors on terminals of somatostatin-containing GABAergic neurons may increase the neurotransmitter release probability, thus leading to greater levels of extracellular somatostatin (Nyitrai et al., 2003). Somatostatin improves LTP in DG (Nakata et al., 1996, see, however, Baratta et al., 2002). Interestingly, the GABAB antagonist CGP36742 appears to selectively affect release of somatostatin, while not affecting release of other neurotransmitters including cholecystokinin, GABA and glutamate (Bonanno et al., 1999). This suggests that presynaptic GABAB receptors on somatostatinergic terminals might have a greater sensitivity to GABAB antagonists than receptors on other terminals. Second, it is reported that treatment with CGP36742 or CGP56433A increased levels of mRNAs for NGF and BDNF (Heese et al., 2000). BDNF is required for development of the late phase of LTP (for review see Bramham and Messaoudi, 2005) and, thus, its up-regulation could be beneficial for long-term synaptic plasticity. A chronic increase in the level of neurotrophic factors may also result in reorganization of synaptic connections. Third, GABAB antagonists increase responsiveness to exogenously applied acetylcholine (Andre et al., 1992), suggesting that suppression of GABAB receptors may augment cholinergic function that, in turn, facilitates tetanus-evoked LTP (Blitzer et. al., 1990; Sokolov and Kleschevnikov, 1995) or induces Thuscarinic' LTP (Segal and Auerbach, 1997). Fourth, suppression of presynaptic GABAB receptors on glutamatergic terminals may increase the release probability of glutamate (Gutovitz et. al., 2001; Waldmeier et al., 2008), which might increase efficiency of excitatory neurotransmission. Fifth, GABAB receptors are linked to activation of ATF4 (Nehring et al., 2000; White et al, 2000; Vernon et al., 2001), a transcription factor that acts to constrain long-term synaptic changes (Chen et al., 2003). Suppressing of GABAB receptors may thus result in a less pronounced activation of ATF4 and better LTP. Whether or not LTP in Ts65Dn is affected through these mechanisms is a subject for future studies.

GABAB antagonists as cognitive enhancers. GABAB receptors are highly expressed in the hippocampus and neocortex (Margeta-Mitrovic et al., 1999; Charles et al., 2001), brain regions critically involved in learning and memory. The impact of the GABAB receptors on neuronal excitability, release of neurotransmitters, and on synaptic plasticity suggests that GABAB receptors may be a target for pharmacological interventions aimed at regulating cognition. Indeed, selective agonists and antagonists of the GABAB receptors affect learning and memory. The GABAB agonist baclofen attenuated anterograde memory in a passive avoidance test (Swartzwelder et al., 1987) and learning in water maze (McNamara and Skelton, 1996; Arolfo et al., 1998). In contrast, GABAB receptor antagonists improved performance in a spatial delayed nonmatch-to-sample task (Staubli et al., 1999; Helm et al., 2005), passive avoidance (Mondadori et al., 1993; Froestl et al., 2004), two-way avoidance test with negative reinforcement (Getova and Bowery, 2001) and social learning (Mondadori et al., 1993; 1996). Certain GABAB antagonists have been evaluated as potential cognitive enhancers (Bullock, 2005; Helm et al., 2005).

GABAB antagonists effects on postsynaptic GABAB/Kir3.2 signaling and LTP in Ts65Dn mice prompted us to study their impact on behavior. We used CGP55845, a high-affinity GABAB antagonist that exhibits behavioral effects after i.p. injections (e.g., Getova and Bowery, 1998; 2001). We found that chronic treatment of Ts65Dn mice with CGP55845 did not change spontaneous locomotor activity or behavior in the open field, but did improve hippocampus-dependent memory. The novel object recognition test, which investigates hippocampus-dependent recognition memory, was markedly affected. Interestingly, improvement of memory in this test was observed only in Ts65Dn mice, a result consistent with the finding that LTP in Ts65Dn slices was more susceptible to treatment with CGP55845. These observations raise the possibility that DS-specific changes of neuronal properties in Ts65Dn mice enhance receptivity to GABAB antagonists.

GABAB receptors and epilepsy: Do GABAB antagonists have an antiepileptic potential in DS? People with DS have a markedly increased risk of epilepsy (Pueschel et al., 1991; Goldberg-Stern et al., 2001; Eisermann et al., 2003; Menendez, 2005). For this reason, any treatment that would reduce inhibitory neurotransmission must be carefully evaluated for its effect on epileptiform activity. Thus, whether or not GABAB antagonists would affect synaptic and neuronal properties relevant to pro-epileptiform activity is an important question. We examined this issue in two sets of experiments. In the first, we investigated GABAB antagonist effects on feedback inhibition by measuring paired-pulse depression of the population spike. Feedback inhibition controls excitability of granule cells and, thus, may impact epileptiform activity. GABAB antagonists increased feedback inhibitory efficiency in the hippocampus of normal rodents (Olpe et al., 1993a). However, because baseline feedback inhibition appears to be stronger in Ts65Dn (Kleschevnikov et al., 2004), we considered the possibility that GABAB antagonists may act differently in this system. In fact, we observed that GABAB antagonists increased paired-pulse depression of population spike in both 2N and Ts65Dn DG; expressed in percent of the baseline values, the changes were similar in 2N and Ts65Dn DG. Thus, GABAB antagonists appear to be equally effective in enhancing feedback inhibition in both the Ts65Dn and 2N DG.

In the second set of experiments we more directly examined ictogenesis in the Ts65Dn DG using a high-potassium model of epilepsy. Because postsynaptic GABAB/Kir3.2 signaling is increased, we were concerned that seizures induced by a high level of extracellular potassium may be exacerbated in the Ts65Dn DG. Indeed, we found that the frequency of spontaneous ictal discharges was greater in Ts65Dn slices. While the GABAB antagonist had no effect on the frequency of ictal bursts, it markedly reduced the amplitude of the related field potentials. This effect was greater in Ts65Dn than in 2N slices. The results thus show that certain properties of inhibitory circuits are not perturbed in the Ts65Dn DG and that GABAB antagonists may have anti-epileptic potential.

Epilepsy is a complex disorder with multiple underlying mechanisms. DS is characterized by an increased incidence of seizures called ‘infantile spasms’, affecting up to 14% of children (Strafstrom, 1993; Strafstrom and Konkol, 1994), and a late-onset seizure' disorder affecting up to 46% of individuals in adulthood (McVicker et al., 1994; Prasher, 1995). The Ts65Dn mouse model of DS was recently proposed as a model for the infantile spasms; it was shown also that GABAB antagonists might ameliorate baclofen-provoked acute epileptic extensor spasms in Ts65Dn (Cortez et al., 2009). Our findings suggest that one possible mechanism for the antiepileptic potential of GABAB antagonists is enhancement of the efficiency of feedback inhibition. A number of studies have described antiepileptic properties of GABAB antagonists in various experimental models (Getova et al., 1998; Canning and Leung, 2000; Czuczwar and Patsalos, 2001). However, in some experimental settings GABAB antagonists provoke seizures (Mares and Slamberová, 2006). Therefore, although our findings suggest that GABAB antagonists have an antiepileptic potential in DS, this property must be tested rigorously in carefully planned clinical trials.

Conclusion. In demonstrating abnormalities in the function of GABAB receptor signaling in Ts65Dn DG, we provide evidence that GABAB antagonists and blocker of Kir3.2 channels improve synaptic plasticity and hippocampus-dependent memory in a mouse model of DS, and that drugs in this class do not appear to increase risk of epilepsy.

The efficacy of the methods set forth above in Ts65Dn DS mice provides a process with widespread clinical utility. Traditionally, MR disorders have been unresponsive to pharmacological interventions, perpetuating the notion that they are treatment-resistant vestiges of abnormal brain development. The data on adult mice, however, indicates that this is not the case. The findings also point to the possibility that mature, but faulty circuits in MR, can be reopened from their present adult configuration and rewired to increase synaptic plasticity.

It is evident from the above results and discussion that improved methods for treating cognitive impairment are provided. The subject methods provide an effective means for improving cognitive function, particularly in individuals suffering from cognitive impairment disorders, e.g., Down syndrome, etc. As such, the subject methods represent an important contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of improving a cognitive function of child or young adult mammal suffering from mental retardation, said method comprising: administering not more than once daily to the child or young adult an effective amount of a pharmaceutical formulation comprising a GABA_{B} receptor antagonist, or a blocker of Kir3.2 potassium channels, wherein cognitive function of the child or young adult is improved.

2. The method of claim 1, wherein the GABA_{B} receptor antagonist is administered orally.

3. The method of claim 1, wherein said GABA_{B} receptor antagonist is delivered parenterally.

4. The method of claim 1, wherein said cognitive function is at least one of learning and memory.

5. The method of claim 4, further comprising the step of testing the child or young adult for improvement in at least one of learning and memory.

6. The method of claim 1, wherein the young adult or child is human.

7. A method of improving a cognitive function of an individual with Down Syndrome, the method comprising: administering not more than once daily to the individual an effective amount of a pharmaceutical formulation comprising a GABA_{B} receptor antagonist; wherein cognitive function of the individual is improved.

8. A method of screening a candidate agent for treatment of cognitive impairment associated with mental retardation, the method comprising: contacting a GABA_{B} receptor with a candidate agent; determining if said agent is an antagonist; administering said agent to an animal in a model for mental retardation; determining if said agent improves cognitive function.
